(19) United States
(12) Patent Application Publication
(10) Pub. No.: US 2020/0377435 A1
(43) Pub. Date: Dec. 3, 2020

(54) Process and Apparatus for Converting Carbon Dioxide Into Methanol

(71) Applicant: Siemens Aktiengesellschaft, München (DE)

(72) Inventors: Katharina Meltzer, Erlangen (DE); Manfred Baldauf, Erlangen (DE); Alexander Tremel, Möhrendorf (DE)

(73) Assignee: Siemens Aktiengesellschaft, München (DE)

(21) Appl. No.: 16/606,806

(22) PCT Filed: Apr. 18, 2018
(86) PCT No.: PCT/EP2018/059871

§ 371 Date: Oct. 21, 2019

(30) Foreign Application Priority Data
Apr. 21, 2017 (DE) 10 2017 206 763.2

(51) Int. Cl. C07C 29/152; C07C 29/151; B01J 8/00

(52) U.S. Cl. CPC C07C 29/152; C07C 29/1514; C07C 29/1516; C07C 31/04; B01J 2208/00893; B01J 2208/00938; B01J 8/008

(57) ABSTRACT

Various embodiments include a process for converting a starting material mixture comprising carbon dioxide and hydrogen into methanol and water. The method may include: providing a catalyst to a reactor; feeding carbon dioxide and hydrogen into the reactor, at a first pressure and a first temperature; feeding a first liquid component into the reactor at a second temperature lower than the first temperature; converting the carbon dioxide and hydrogen into methanol and water; removing the methanol from a gas phase in at least one liquid phase comprising the first component and the water; and discharging a first liquid material stream comprising the first component, methanol, and water from the reactor. At the first temperature and the first pressure the methanol is in a gaseous state and the first component and the water are in a liquid state.

### CROSS-REFERENCE TO RELATED APPLICATIONS

This application is a U.S. National Stage Application of International Application No. PCT/EP2018/059871 filed Apr. 18, 2018, which designates the United States of America, and claims priority to DE Application No. 10 2017 206 763.2 filed Apr. 21, 2017, the contents of which are hereby incorporated by reference in their entirety.

### TECHNICAL FIELD

The present disclosure relates to chemical treatment of carbon dioxide. Various embodiments may include processes and/or apparatus for converting carbon dioxide and hydrogen into methanol and water.

### BACKGROUND

The demand for electrical power fluctuates substantially over the course of the day. With a growing proportion of power now obtained from renewable energy sources, power generation fluctuates over the course of the day too. To offset the oversupply of power during periods with plenty of sun and strong wind but low demand for power, regulatable power plants or storage facilities are needed in order to store this energy. One proposed solution is the conversion of electrical energy into usable products that may be used in particular as platform chemicals. One possible technique for the conversion of electrical energy into usable products is electrolysis. Electrolysis of water into hydrogen and oxygen in particular is known.

Carbon dioxide is a climate-damaging greenhouse gas. It contributes significantly to the greenhouse effect and consequently to global heating. The reduction of carbon dioxide emissions, particularly in industrial processes, is therefore desirable. In order for the operation of different industrial processes to be as climate-neutral as possible, i.e. with low emission of carbon dioxide, it is desirable to convert the carbon dioxide formed in these processes into usable substances.

The hydrogen produced in electrolysis can be reacted with climate-damaging carbon dioxide to form the usable product methanol. Because of the reaction thermodynamics, the conversion at equilibrium in the reaction of carbon dioxide and hydrogen to methanol and water is severely limited. The starting material used in conventional methanol synthesis is synthesis gas, i.e. a mixture of hydrogen, carbon monoxide, and carbon dioxide. Harsh reaction conditions of 50-100 bar and 200-300° C. are needed to carry out this reaction.

If predominantly carbon dioxide and hydrogen are used as starting material instead of synthesis gas, the conversion rates achieved are much lower compared with the production of methanol from synthesis gas. The conversion of carbon dioxide under these reaction conditions is disadvantageously of the low order of about 20%. In order to increase the overall conversion of hydrogen and carbon dioxide into methanol, the unreacted carbon dioxide and unreacted hydrogen are recirculated. Because pressure losses occur in the pipelines used and in the reactor, a compressor to recompress the recirculated and unreacted gases is necessary. The greater the amount of gas mixture that is recirculated, the higher, and thus more disadvantageous, the energy requirement for the conversion of carbon dioxide into methanol, since the compressor requires large amounts of energy.

### SUMMARY

The teachings of the present disclosure describe example processes and/or apparatus that increase conversion in the reaction of hydrogen and carbon dioxide to afford methanol and water and are energy efficient in accomplishing this. For example, some embodiments include a process for converting a starting material mixture comprising carbon dioxide (CO₂) and hydrogen (H₂) into a first product, methanol, (MeOH) and a second product, water (H₂O), having the following steps: providing a reactor (2) containing a catalyst, feeding carbon dioxide (CO₂) and hydrogen (H₂) into the reactor (2), wherein the reactor (2) is at a first pressure (P1) and a first temperature (T1), feeding a first liquid component (C1) as coolant and as an agent for removing the first product, methanol (MeOH), into the reactor (2), wherein the first liquid component (C1) is at a second temperature (T2) that is lower than the first temperature (T1), converting the carbon dioxide (CO₂) and hydrogen (H₂) into the first product, methanol (MeOH), and the second product, water (H₂O), wherein the first temperature (T1) and the first pressure (P1) in the reactor (2) are set so that the methanol (MeOH) is in the gaseous state and the first component (C1) and the second product, water (H₂O), are liquid, removing the first gaseous product, methanol (MeOH), from a gas phase in at least one liquid phase comprising the first component (C1) and the second product, water (H₂O), and discharging a first liquid material stream comprising the first component (C1), the first product, methanol (MeOH), and the second product, water (H₂O), from the reactor (2).

In some embodiments, removal takes place through condensation and/or absorption.

In some embodiments, the first material stream is fed into a separator (3) and the first product, methanol (MeOH), is separated in the separator (3).

In some embodiments, the first liquid component (C1) is water.

In some embodiments, the first liquid component (C1), water, and the second product, water (H₂O), are separated in the separator (3) and the water is fed back into the reactor (2) as the first liquid component (C1).

In some embodiments, the first component (C1) is a fuel.

In some embodiments, the fuel is gasoline, diesel or an alcohol.

In some embodiments, the mass ratio of the first component (C1) to methanol (MeOH) is in a range from 2:1 to 5:1.

In some embodiments, the mass ratio of the first component (C1), water, to methanol (MeOH) is 3:1.

In some embodiments, the first pressure (P1) is in a range between 100 bar and 300 bar.

In some embodiments, the first temperature (T1) is in a range between 200° C. and 300° C.

In some embodiments, the second temperature (T2) is in a range between 5° C. and 100° C.

As another example, some embodiments include a reaction apparatus (1) for executing a process as claimed in any of claims 1 to 12 comprising: a reactor (2) for converting carbon dioxide (CO₂) and hydrogen (H₂) into a first product, water (H₂O), and a second product, methanol (MeOH), a first inlet for feeding the carbon dioxide (CO₂) and hydrogen (H₂) into the reactor (2), a second inlet for feeding a first liquid component (C1) into the reactor (2), a first outlet for discharging a first liquid material stream comprising the first and second product (MeOH, H₂O) and the first component (C1), and a first holding tank containing the first liquid component (C1), which is connected to the second inlet through a first pipeline.

In some embodiments, there is a thermal separator (3).

In some embodiments, the thermal separator is connected to the first holding tank and/or to the reactor (2) through a second pipeline carrying water.

### BRIEF DESCRIPTION OF THE DRAWINGS

Further features, characteristics, and advantages of the teachings of the present disclosure are given in the description below, with reference to the accompanying figures. In these schematic representations:

FIG. 1 shows an apparatus having a reactor for converting carbon dioxide and hydrogen into methanol and water, with addition of water as the first component, and a thermal separator, incorporating teachings of the present disclosure;

FIG. 2 shows a graph of the methanol yield as a function of pressure for different first temperatures in the presence and absence of water; and

FIG. 3 shows an apparatus having a reactor for converting carbon dioxide and hydrogen into methanol and water, with addition of ethanol as the first component, and a phase separator, incorporating teachings of the present disclosure.

### DETAILED DESCRIPTION

Some embodiments include a process for converting carbon dioxide and hydrogen into a first product, methanol, and a second product, water, comprises firstly the provision of a reactor with a catalyst. Carbon dioxide and hydrogen are fed into the reactor. The conditions in the reactor comprise a first pressure and a first temperature. The reactor is additionally charged with a first liquid component as coolant and as an agent for removing the first product, methanol. The first liquid component is here at a second temperature that is lower than the first temperature. The first liquid component is therefore able to cool the reactor. The conversion of carbon dioxide and hydrogen into the first product, methanol, and the second product, water, takes place in the reactor, with the first temperature and first pressure in the reactor set so that the methanol is at least partly in the gaseous state and the first component and second product, water, are at least partly liquid. The first gaseous product, methanol, can then be removed from the gaseous phase by means of the at least one liquid phase comprising the first component and the second product, water. A first liquid material stream comprising the first component, methanol, and the second product, water, is discharged from the reactor.

In some embodiments, an apparatus incorporating teachings of the present disclosure for the execution of the process for converting carbon dioxide and hydrogen into a first product, methanol, and a second product, water, comprises a reactor with a catalyst. The apparatus additionally comprises a first inlet into the reactor for feeding of the carbon dioxide and hydrogen to the reactor. The apparatus additionally comprises a second inlet into the reactor for feeding of the first liquid component. The apparatus also comprises a first outlet from the reactor for discharging the first material stream comprising the first component methanol and the second product, water. The apparatus according to the invention also comprises a holding tank filled with the first liquid component, which is connected to the reactor through a first pipeline.

Gaseous methanol is defined herein as both methanol that is in the vapor state, that is to say condensable, and methanol present as a supercritical fluid. If the methanol is present in the vapor state, its removal from the gas phase occurs through condensation. If, on the other hand, the methanol is present as a supercritical fluid, its removal from the gas phase occurs through absorption into the first liquid component and/or into the second product, water.

Within the reactor, a thermodynamic equilibrium is reached between the gas phase and the liquid phase. This means that the second product, water, and the first component are present in the gas phase only in small amounts on account of the thermodynamic equilibrium.

The continuous removal from the reactor of the product-containing liquid phase results in the reaction equilibrium being shifted to the product side, thereby increasing conversion. The degree of conversion of the carbon dioxide and hydrogen into methanol may be increased considerably with this process. With some embodiments, it is even possible to achieve almost complete conversion. In some embodiments, it is then no longer necessary to recirculate the gas phase, which means that it is no longer necessary to compress the gas phase. This results in the process being energy-efficient and achieving a higher conversion.

In addition, the reaction of hydrogen and carbon dioxide to afford methanol and water has a high enthalpy of reaction. This is a strongly exothermic reaction in which a considerable amount of heat is evolved. If the mixture of starting materials contains not only carbon dioxide but also a typically small amount of carbon monoxide, there is an additional increase in the exothermicity of the reaction. The increase in conversion means that the amount of heat evolved in the reaction increases still further. The condensation of the methanol results in a further evolution of heat. Cooling of this process is therefore necessary. Through the choice of the second temperature of the first liquid component, that is to say a lower second temperature compared with the first temperature, it is possible to cool this reaction and prevent a high evolution of heat. The cooling of the reaction may prolong the lifetime of the catalyst, which typically tends to become deactivated at high temperatures.

The addition of the first component at a second temperature below the first temperature allows the condensation of the methanol to be carried out even at lower pressures. In some embodiments, the energy efficiency is thus further boosted by the lower pressure conditions in the reactor allied with increased conversion compared with the conventional reaction conditions.

Methanol synthesis typically uses catalysts based on Cu—ZnO—Al₂O₃ or Cr₂O₃ containing various additives and promoters. The high-pressure methanol production process known from the prior art uses catalysts based on ZnO—Cr₂O₃ (without copper). New catalysts based on gold Au, silver Ag, palladium Pd or platinum Pt are currently being developed, but are not commercially viable, as they are costly while providing only small improvements in yield.

In some embodiments, the first material stream is fed into a thermal separator and the first product, methanol, is separated from the second product, water, in the thermal separator. Thus, in addition to the reactor, the reaction apparatus in this case comprises a thermal separator too. This yields methanol that can be used as a starting material for further reactions. In some embodiments, it may be converted into dimethyl ether or oxymethylene ether, both of which are substances that may be used as fuels. The methanol may in particular be used directly as a fuel or mixed into fuels, particularly diesel.

In some embodiments, the first liquid component is water. This water mixes with the second product, water, and acts as an agent for removing the methanol from the gas phase. The added water also acts here as a coolant for the reaction. In some embodiments, the water is separated from the methanol in the thermal separator.

This affords the possibility of feeding the separated water back into the reactor as the first liquid component. The water may be held in a holding tank, with the holding tank connected to the reactor through a pipeline, thereby allowing the first liquid component to be fed from the holding tank into the reactor. The water separated in the thermal separator can then either be fed back directly into the reactor or fed back into the holding tank.

In some embodiments, the mass ratio of water, which is added as the first component, to methanol, the first product, may be three to one. This means that if, in particular, 3 kg of water is fed into the reactor, 1 kg of methanol can be produced and removed into the liquid phase, with this process being almost isothermal. This means that no additional cooling of the reactor is necessary.

The capacity of the water to absorb heat at a constant second temperature, i.e. the temperature at which the first liquid component is admitted to the reactor, increases with increasing reaction temperature, which means that the amount of water needed to remove 1 kg of methanol from the gas phase decreases.

In some embodiments, the first liquid component is a fuel. In some embodiments, the fuel used may be gasoline, diesel or an alcohol. These fuels themselves constitute a proportion of a possible end product. An end product may in particular be a fuel enriched with methanol. The fuel end product in this case contains a defined proportion of methanol. In this realization, the fuel is fed into the reactor as the first liquid component, thereby serving both as coolant and as the agent for removing the methanol. If the methanol is present as a supercritical fluid, it is absorbed into the liquid phase, which comprises predominantly fuel.

In some embodiments, the liquid phase also contains the second product, water. In technical uses this is often undesirable. To separate the water, distillation of the liquid material stream in particular may then be carried out. If the fuel is completely miscible with methanol, but not with the second product, water, a two-phase mixture forms, particularly after being discharged from the reactor and particularly if there is an additional reduction in temperature outside the reactor relative to the internal reactor temperature. One phase consists largely of water and methanol. The other phase consists largely of methanol and the fuel. If an alcohol is used as the fuel, these are especially advantageously ethanol, butanol or hexanol.

In some embodiments, the fuel has good miscibility with methanol and water at elevated temperature, in particular at the internal temperature in the reactor, i.e. the first temperature, in particular a temperature of at least 100° C. This results in good absorption of methanol and the second product, water, under the reaction conditions in the reactor. If the first liquid material stream, which in this case comprises the fuel, the methanol, and the second product, water, is discharged from the reactor and cooled to temperatures below 100° C., a miscibility gap results in the formation of a two-phase mixture. In some embodiments, a water phase forms here that has only a very low methanol content. Longer-chain alcohols, in particular butanol or hexanol, have particularly suitable properties for optimal partition of the methanol between the two liquid phases, e.g. mostly in the fuel-rich phase. In this case, distillative processing of the fuel end product may be carried out with a lower number of theoretical plates or distillative processing may be dispensed with altogether.

In some embodiments, the mass ratio of the first liquid component to methanol is in a range between 2:1 and 5:1. In some embodiments, these amounts of the liquid components are sufficient for the heat evolved in the reactor from condensation and from the reaction to be conducted out of the reactor, thereby allowing the reactor to be operated almost isothermally. In some embodiments, this means that the use of a costly external cooling unit may be avoided and the energy efficiency thus increased.

In some embodiments, the first pressure is in a range of 100 bar to 250 bar. In some embodiments, the first pressure is as low as possible within this range. This may increase the energy efficiency of the process. However, the set pressure must also be high enough to allow the reaction of carbon dioxide to afford the first product, methanol, with adequately high conversion. Adequately high conversion is considered to be conversion in a range of 50% to 80% per turnover cycle. In some embodiments, this process allows almost complete conversion to be achieved, which means that the presence of a recycle compressor to compress unreacted starting materials before feeding back into the reaction apparatus can be avoided.

In some embodiments, the first temperature is in a range between 200° C. and 300° C. The critical temperature of methanol is 240° C. The critical temperature of water is 374° C. In the stated temperature range, water is therefore in the vapor state, which means that the second product, water, can condense. It accordingly serves as an additional agent for removing methanol from the gas phase.

In some embodiments, the second temperature of the first liquid component is below the reaction temperature, in particular in a range between 5° C. and 100° C. In this temperature range it is, in particular, possible for the heat evolved in the reactor to be completely conducted out of the reactor by the first liquid component. In some embodiments, it is thus possible for the reaction to be conducted isothermally without use of additional external cooling.

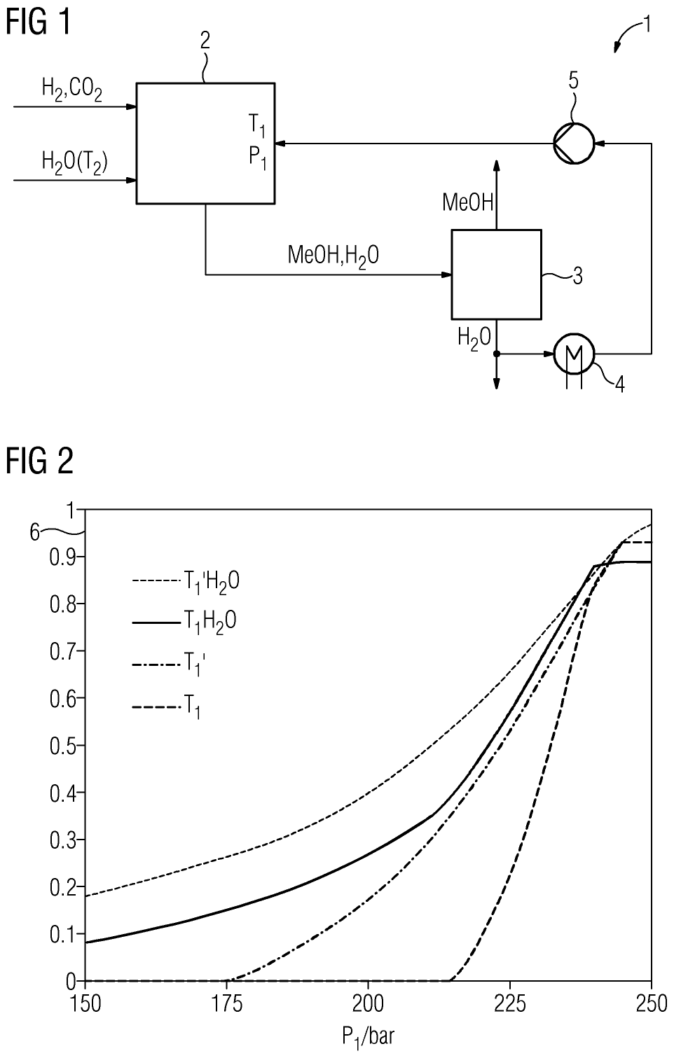

FIG. 1 shows an apparatus 1 having a reactor 2 for converting carbon dioxide CO₂ and hydrogen H₂ into methanol MeOH and water H₂O. The apparatus 1 also includes a thermal separator 3.

Firstly, a starting material mixture comprising hydrogen H₂ and carbon dioxide CO₂ is fed into the reactor 2. The starting material mixture comprising carbon dioxide CO₂ and hydrogen H₂ may also contain a small proportion of carbon monoxide. Compared to the usual methanol synthesis from carbon monoxide, this proportion is, however, considerably lower. However, during the reaction a certain amount of carbon monoxide CO and water H₂O is formed from carbon dioxide CO₂ and hydrogen H₂ through the water-gas shift reaction. The percent carbon monoxide content is typically in the low single-figure range of less than 5%, depending on the catalyst.

The conditions in the reactor comprise a first temperature T1 and a first pressure P1. The first temperature T1 in this example is 250° C. and the first pressure in this example is 240 bar. H₂ and CO₂ are typically compressed in a compressor (not shown in FIG. 1), which is connected upstream of reactor 2. In addition, reactor 2 contains a catalyst that catalyzes the reaction of carbon dioxide with hydrogen to afford methanol and water.

Methanol synthesis typically uses catalysts based on Cu—ZnO—Al₂O₃ or Cr₂O₃ containing various additives and promoters. New catalysts based on gold Au, silver Ag, palladium Pd or platinum Pt are currently being developed, but are not commercially viable, as they are costly while providing only small improvements in yield.

This example uses water H₂O as the first liquid component C1. The water is added from reactor 2. The water may be sprayed into the reactor. In some embodiments, the water is added close to the catalyst, to provide cooling to the latter. The second temperature T2 of the added water H₂O is lower than the reactor temperature. In this example it is in particular 50° C. Within the reactor, the first product, methanol MeOH, and the second product, water H₂O, are produced from hydrogen H₂ and carbon dioxide CO₂. Under these conditions, the methanol MeOH is absorbed into the liquid water. The liquid water is typically present at the bottom of the reactor. The liquid mixture of water H₂O, which originates from the reaction as the second product and as the water added as the first liquid component, and the methanol MeOH are discharged from the reactor as a liquid material stream. The liquid material stream is fed into a thermal separator 3.

In this example, the thermal separator is a distillation apparatus. However, other thermal separation processes may also be used, in particular pervaporation, adsorptive processes, or other membrane processes. In the distillation apparatus 3, methanol MeOH and water H₂O are separated from one another. The water H₂O is fed back into reactor 2 at least in part. The water is cooled in a first condenser 4. The cooled water is pumped back into the reactor by means of a circulating pump 5. In some embodiments, the water may be fed into a holding tank, in which the fresh water is held prior to the reaction, and then subsequently fed into the reactor together with the first liquid component water.

FIG. 2 shows a graph plotting the yield of methanol in the liquid phase 6 against the first pressure P1. The yield here is defined as the molar flow of methanol in the liquid phase 6 relative to the molar flow of carbon dioxide in the reactor 2. The yield is plotted as a function of the pressure P1 for two different temperatures, a first temperature T1 of 250° C. and a first temperature T1′ of 225° C. The reaction takes place here in the gas phase. The graph shows that, at low pressures, the yield is virtually 0. Only at pressures above respectively 175 bar and 200 bar does the yield 6 start to rise.

The graph also shows the yield at the first temperature T1 and the first temperature T1′ when water is added as a first component at a second temperature of 50° C. The molar amount of water added to reactor 2 is twice that of the hydrogen. It is clear that when water is fed into the reactor as a first liquid component, the yield increases markedly, even at lower pressures. This demonstrates that, even without the liquid material stream being fed out of the reactor, the yield is increased by the addition of water, since the first product, methanol MeOH, is absorbed by the water. The continuous discharging of the methanol-water mixture means that the product is being continuously removed from the gas phase and then fed out of the reactor. The reaction equilibrium is therefore shifted to the product side, which increases the yield. In addition, the discharging of the products from the reactor results in the reaction equilibrium at constant yield being shifted in favor of lower pressures. For reactor 2, this means that a lower first temperature T1 and a lower first pressure P1 can be chosen than would have been the case without the addition of water, with this even being accompanied by an advantageous increase in yield.

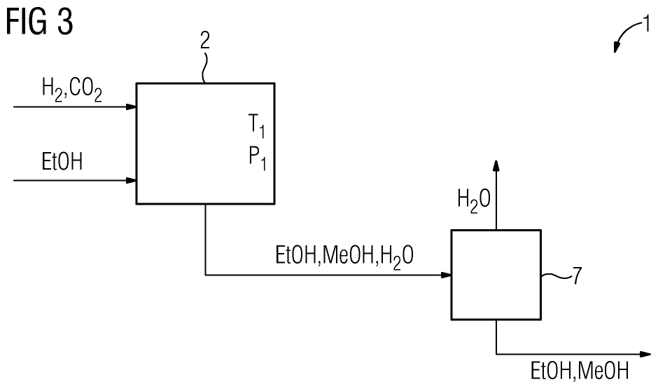

FIG. 3 shows a second working example in which, into apparatus 1, ethanol is fed into the reactor 2 as a first component. As in the first working example, hydrogen H₂ and carbon dioxide CO₂ are fed into the reactor 2. The conditions in the reactor comprise a first pressure P1 and a first temperature T1. In the gas phase, hydrogen H₂ and carbon dioxide CO₂ react to form methanol MeOH and water H₂O. The methanol MeOH is absorbed by the ethanol EtOH and the second product, water H₂O, condenses. The first material stream comprising ethanol EtOH, methanol MeOH, and water H₂O, is discharged continuously from the reactor 2. This results in the reaction being shifted to the product side, which increases the yield of methanol MeOH considerably. The end product in this example is a fuel that comprises ethanol and methanol, but no water.

The water must therefore be separated in separator 7. As in the first working example, the separator 7 is a distillation apparatus. For the use of a longer-chain alcohol as first liquid component 3, it is possible to replace the distillation apparatus 7 with a phase separator. By setting a lower temperature, the separation from the water of the longer-chain alcohol and the methanol MeOH as an organic phase can be exploited to obtain two liquid phases. If the partitioning of the methanol MeOH between the alcohol EtOH and the water H₂O is such that it is present predominantly in the alcohol phase, then the alcohol-containing phase may be separated in the phase separator. This reduces the energy consumption of the separation, which means that the requisite number of theoretical plates for the distillation column can be lower, thus reducing capital costs. In some embodiments, it is even possible to avoid using a distillation process altogether.

The ratio of added ethanol to condensed methanol in the liquid phase is 4 to 1. This means that 4 kg of ethanol needs to be used in order to obtain 1 kg of methanol in the liquid phase. In some embodiments, with this ratio it is also possible to dissipate the heat evolved in the reaction. This allows an additional cooling unit in reactor 2 to be avoided. In some embodiments, the ethanol, or the water in the case of the first working example, is sprayed close to the catalyst, to allow the heat of reaction evolved to be directly dissipated in situ.

What is claimed is:

1. A process for converting a starting material mixture comprising carbon dioxide and hydrogen into a first product, methanol, and a second product, water, the method comprising:
providing a catalyst to a reactor;
feeding carbon dioxide and hydrogen into the reactor, wherein the reactor is at a first pressure and a first temperature;
feeding a first liquid component into the reactor as coolant and as an agent for removing the first product, wherein the first liquid component is fed at a second temperature lower than the first temperature;
converting the carbon dioxide and hydrogen into the first product, and the second product, wherein, at the first temperature and the first pressure the methanol is in a gaseous state and the first component and the second product are in a liquid state;
removing the methanol from a gas phase in at least one liquid phase comprising the first component and the second product; and
discharging a first liquid material stream comprising the first component, the first product, and the second product from the reactor.

2. The process as claimed in claim 1, wherein removal takes place through condensation and/or absorption.

3. The process as claimed in claim 1, further comprising:
feeding the first material stream into a separator; and
separating the first product in the separator.

4. The process as claimed in any of claim 1, wherein the first liquid component comprises water.

5. The process as claimed in claim 1, further comprising:
separating the first liquid component and the second product in a separator; and
feeding water is back into the reactor as the first liquid component (C1).

6. The process as claimed in any of claim 1, wherein the first component comprises a fuel.

7. The process as claimed in claim 6, wherein the fuel comprises at least one of gasoline, diesel, or an alcohol.

8. The process as claimed in claim 1, wherein a mass ratio of the first component to methanol is in a range from 2:1 to 5:1.

9. The process as claimed in claim 4, wherein a mass ratio of the first component to methanol is 3:1.

10. The process as claimed in claim 1, wherein the first pressure is in a range between 100 bar and 300 bar.

11. The process as claimed in claim 1, wherein the first temperature is in a range between 200° C. and 300° C.

12. The process as claimed in claim 1, wherein the second temperature is in a range between 5° C. and 100° C.

13. A reaction apparatus comprising:
a reactor for converting carbon dioxide and hydrogen into water and methanol;
a first inlet for feeding the carbon dioxide and hydrogen into the reactor;
a second inlet for feeding a first liquid component into the reactor;
a first outlet for discharging a first liquid material stream comprising water and methanol and the first liquid component; and
a first holding tank containing the first liquid component, connected to the second inlet through a first pipeline.

14. The reaction apparatus as claimed in claim 13, further comprising thermal separator.

15. The reaction apparatus as claimed in claim 14, wherein the thermal separator is connected to the first holding tank and/or to the reactor through a second pipeline carrying water.
